(19) United States
(12) Patent Application Publication
(10) Pub. No.: US 2025/0121017 A1
(43) Pub. Date: Apr. 17, 2025

(54) ONCOLYTIC VIROTHERAPY COMPOSITIONS AND METHODS

(71) Applicant: UNM RAINFOREST INNOVATIONS, Albuquerque, NM (US)

(72) Inventor: Eric C. Bartee, Albuquerque, NM (US)

(21) Appl. No.: 18/704,248

(22) PCT Filed: Oct. 27, 2022
(86) PCT No.: PCT/US2022/048013

§ 371 Date: Apr. 24, 2024

Related U.S. Application Data
(60) Provisional application No. 63/302,183, filed on Jan. 24, 2022.
(60) Provisional application No. 63/273,551, filed on Oct. 29, 2021.

(51) Int. Cl. A61K 35/768; A61K 45/06; A61P 35/00; C07K 14/54; C07K 14/705; C07K 16/24; C12N 7/00

(52) U.S. Cl. CPC A61K 35/768; A61K 45/06; A61P 35/00; C07K 14/5434; C07K 14/70521; C07K 16/241; C12N 7/00; C12N 2710/24033

(57) ABSTRACT

In one aspect, composition generally includes a virotherapeutic agent and an inhibitor of tumor necrosis factor (TNF). In one or more embodiments, the virotherapeutic agent can include an oncolytic virus. In one or more embodiments, the inhibitor of TNF comprises can include an antibody, or a fragment thereof, that binds to TNF. In another aspect, a method of treating a tumor in a subject generally includes co-administering to the subject an oncolytic virotherapeutic agent and an inhibitor of tumor necrosis factor (TNF). In one or more embodiments, the oncolytic virotherapeutic agent is administered intratumorally. In one or more embodiments, the inhibitor of TNF is administered systemically.

### CROSS-REFERENCE TO RELATED APPLICATION

This application claims the benefit of U.S. Provisional Patent Application No. 63/273,551, filed Oct. 29, 2021, and U.S. Provisional Patent Application No. 63/302,183, filed Jan. 24, 2022, each of which is incorporated herein by reference in its entirety.

### GOVERNMENT FUNDING

This invention was made with government support under AI142387 and CA194090 awarded by the National Institutes of Health. The government has certain rights in the invention.

### SUMMARY

This disclosure describes, in one aspect, a composition that generally includes a virotherapeutic agent and an inhibitor of tumor necrosis factor (TNF).

In one or more embodiments, the virotherapeutic agent can include an oncolytic virus. In some of these embodiments, the virotherapeutic agent can be a myxoma virus.

In one or more embodiments, the inhibitor of TNF comprises can include an antibody, or a fragment thereof, that binds to TNF.

In another aspect, this disclosure describes a method of treating a tumor in a subject. Generally, the method includes combining the oncolytic virotherapeutic agent with an inhibitor of tumor necrosis factor (TNF). In one or more embodiments, the inhibitor of tumor necrosis factor may be separately co-administered with the oncolytic virotherapeutic agent. In one or more other embodiments, the inhibitor of tumor necrosis factor may be encoded by and expressed from the oncolytic virotherapeutic agent.

In one or more embodiments, the oncolytic virotherapeutic agent is administered intratumorally.

In one or more embodiments, the inhibitor of TNF is administered systemically.

In one or more embodiments, the method further includes administering to the client an additional tumor therapy. In some of these embodiments, the additional tumor therapy comprises chemotherapy, radiation therapy, immunotherapy, or a prodrug, targeted therapy.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Exemplary vPD1/IL 12 therapy in a normally ‘responsive’ lung cancer model. (A) Schematic of experimental design. A9F1 lung cancer cells were implanted into both flanks of syngeneic mice. Once tumors reached ˜30 mm², the larger tumor was treated with three intratumoral injections of vPD1/IL12 or various controls (mock, vPD1, or vIL12). (B) The progression of treated (injected) and untreated (contralateral) tumors (mock, left; vPD1, second from left; vIL12, second from right; vPD1/IL12, right) was then monitored and animals were euthanized when their total tumor burden exceeded 400 mm². (C) Overall survival. Data is included as a reference point to demonstrate the efficacy of vPD1/IL12 in a ‘responsive’ model.

FIG. 2. Exemplary vPD1/IL 12 therapy in a normally ‘responsive’ ovarian cancer model. (A) Schematic of experimental design. BR5 ovarian cancer cells were implanted directly into the peritoneal cavity of syngeneic mice. Ten days after implantation, animals were treated with three intratumoral injections of either saline or vPD1/IL12. (B) The progression of disease was monitored using luciferase imaging and animals were euthanized when their body condition deteriorated to a predetermined level. (C) Overall survival. Data is included as a second reference point to demonstrate the efficacy of vPD1/IL12 in a ‘responsive’ model.

FIG. 3. Exemplary vPD1/IL12 therapy in a normally ‘non-responsive’ melanoma model. (A) Schematic of experimental design. B16/F10 melanoma cells were implanted into both flanks of syngeneic mice. Once tumors reached ˜30 mm², the larger tumor was treated with three intratumoral injections of vPD1/IL12 or various controls (mock, vPD1, or vIL12). (B) The progression of treated (top) and untreated (bottom) tumors (mock, left; vPD1, second from left; vIL12, second from right; vPD1/IL12, right) was then monitored and animals euthanized when their total tumor burden exceeded 400 mm². (C) Overall survival. Data is included as a reference point to demonstrate the normal lack of efficacy of vPD1/IL 12 in a ‘non-responsive’ model.

FIG. 4. Exemplary vPD1/IL12 therapy in a normally ‘non-responsive’ colon cancer model. (A) Schematic of experimental design. MC38 colon cancer cells were implanted into both flanks of syngeneic mice. Once tumors reached ˜30 mm², the larger tumor was treated with three intratumoral injections of vPD1/IL12 or various controls (mock, vPD1, of vIL12). (B) The progression of both the treated (top and non-treated (bottom) tumors was then monitored and animals euthanized when their total tumor burden exceeded 400 mm². (C) Overall survival. Data is included as a second reference point to demonstrate the normal lack of efficacy of vPD1/IL12 in a ‘non-responsive’ model.

FIG. 5. Toxicity associated with vPD1/IL 12 therapy. (A) Schematic of experimental design. B16/F10 melanoma cells were implanted into both flanks of syngeneic mice. Once tumors reached ˜30 mm², the larger tumor was treated with three intratumoral injections of vPD1/IL12 or various controls (mock, vGFP). (B) Body weight of each animal was measured every other day. Data is presented as the percent of each animals starting weight. Note the transient decrease in animal body weight in vPD1/IL 12 treated mice around days 5-10. Data is included as a reference point to demonstrate the toxicity of vPD1/IL12 therapy.

FIG. 6. IFNγ has no effect on vPD1/IL 12 treatment in two models. (A) Schematic of experimental design. B16/F10 melanoma cells were implanted into both flanks of syngeneic mice. Once tumors reached ˜30 mm², the larger tumor was treated with three intratumoral injections of vPD1/IL12. Mice were also given intraperitoneal injection of either isotype or anti-IFNγ antibodies twice weekly throughout the duration of the experiment. (B) Overall survival. (C) Body weight of each animal was measured every other day. Data is displayed as the average body weight for all animals in each cohort.

FIG. 7. IFNγ has no effect on vPD1/IL 12 treatment in two models. (A) Schematic of experimental design. B16/F10 melanoma cells were implanted into both flanks of either WT or IFNγR1-KO mice. Once tumors reached ˜30 mm², the larger tumor was treated with three intratumoral injections of vPD1/IL12. (B) Overall survival. (C) Body weight of each animal was measured every other day. Data is displayed as the average body weight for all animals in each cohort. Data is included as a reference point to demonstrate that elimination of IFNγ (a known immunotherapy effector molecule) does not mimic the results seen with elimination of TNF.

FIG. 8. TNF both promotes toxicity and inhibits efficacy of vPD1/IL12 treatment in a melanoma model. (A) Schematic of experimental design. B16/F10 melanoma cells were implanted into both flanks of either WT or TNF-KO mice. Once tumors reached ˜30 mm², the larger tumor was treated with three intratumoral injections of vPD1/IL12. (B) Body weight of each animal was measured every other day. Data is displayed as the average body weight for all animals in each cohort. (C) Overall Survival. Data is included as a reference point to demonstrate that loss of TNF has several beneficial effects on vPD1/IL12 therapy.

FIG. 9. TNF both promotes toxicity and inhibits efficacy of vPD1/IL 12 treatment in a colon cancer model. (A) Schematic of experimental design. MC38 colon cancer cells were implanted into both flanks of either WT or TNF-KO mice. Once tumors reached ˜30 mm², the larger tumor was treated with three intratumoral injections of vPD1/IL12. (B) Body weight of each animal was measured every other day. Data is displayed as the average body weight for all animals in each cohort. (C) Overall Survival. Data is included as a second reference point to demonstrate that loss of TNF has several beneficial effects on vPD1/IL 12 therapy.

FIG. 10. TNF inhibition improves safety and enhances the efficacy of vPD1/IL12 treatment. (A) Schematic of experimental design. B16/F10 melanoma cells were implanted into both flanks of syngeneic mice. Once tumors reached ˜30 mm², the larger tumor was treated with three intratumoral injections of vPD1/IL12. Mice were also given an intraperitoneal injection of either isotype control or two distinct clones of anti-TNF antibodies (the TN3 clone or the XT3 clone) every other day throughout the duration of the experiment. Progression of both the injected and contralateral tumors was then monitored every other day and mice were euthanized when their total tumor burden exceeded 400 mm². (B) Overall survival of animals. Note that animals treated with vPD1/IL 12 and the XT3 clone displayed improved overall survival. Significance determined by Log-Rank test (**=p<0.01 from all other groups). (C) Body weight of each animal was measured every other day. Data is displayed as the average body weight for all animals in each cohort. Note the loss of body weight following vPD1/IL 12 treatment while animals treated with vPD1/IL12 and the TN3 clone maintain their weight. Data is included as a reference point to demonstrate that therapeutic combination treatment with an oncolytic virus and TNF inhibition demonstrates clinical synergy. Significance determined by Student's T-test (*=p<0.05 between the indicated groups). This figure supports the results shown in FIG. 10.

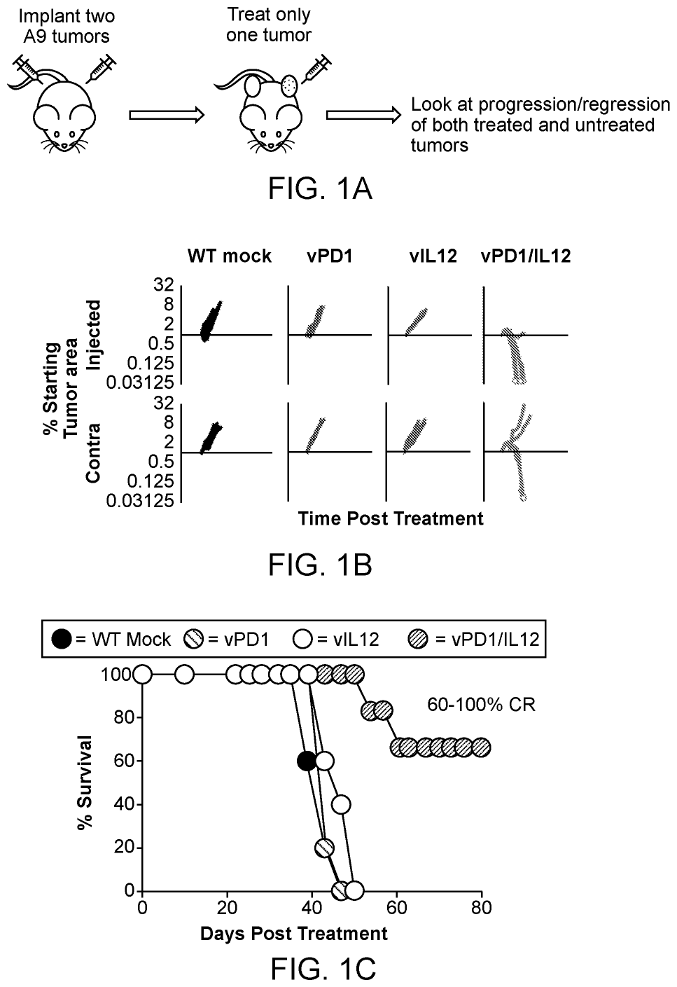

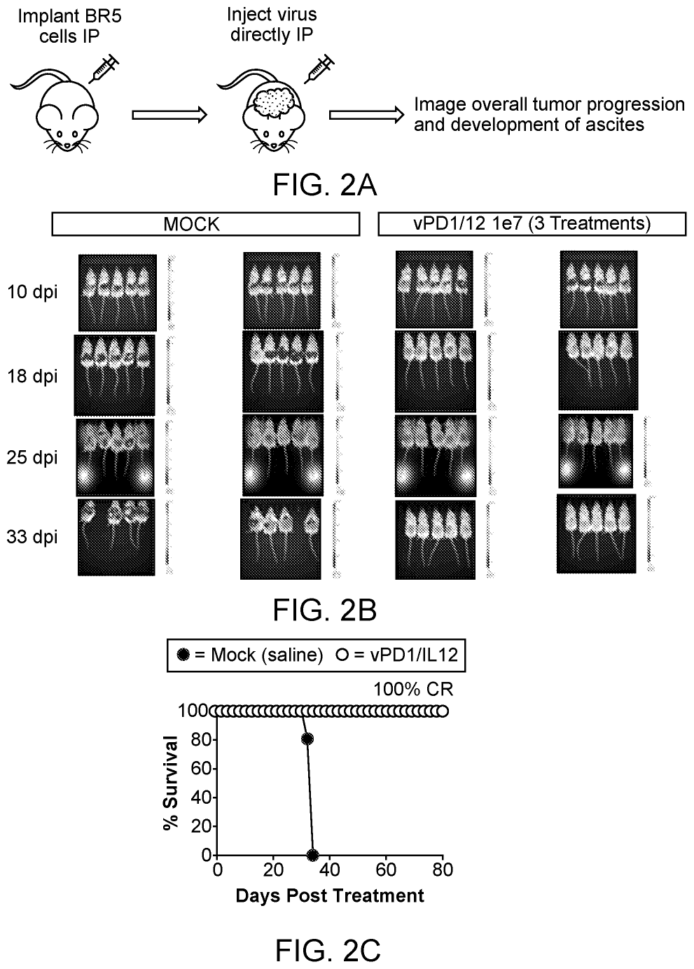

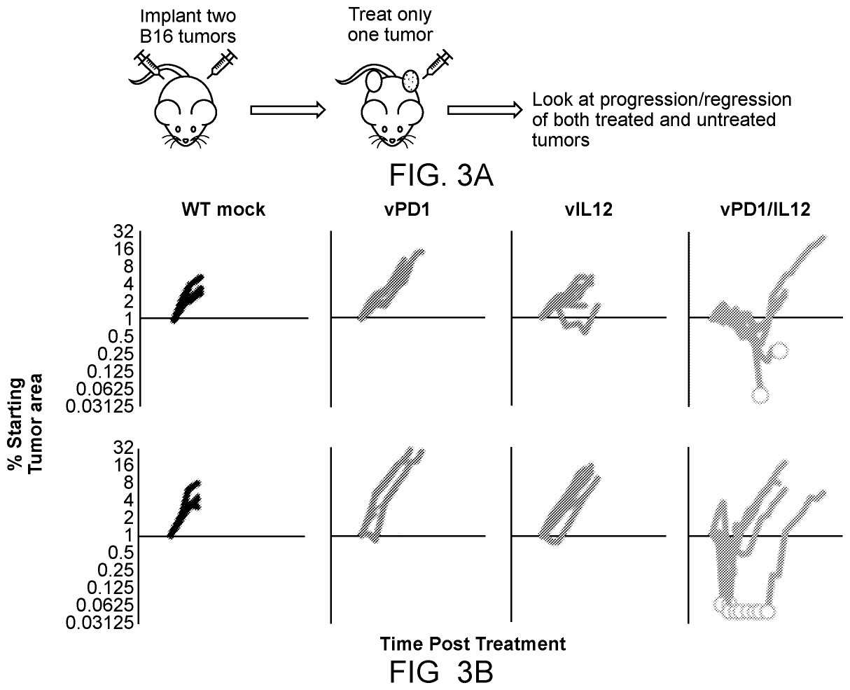

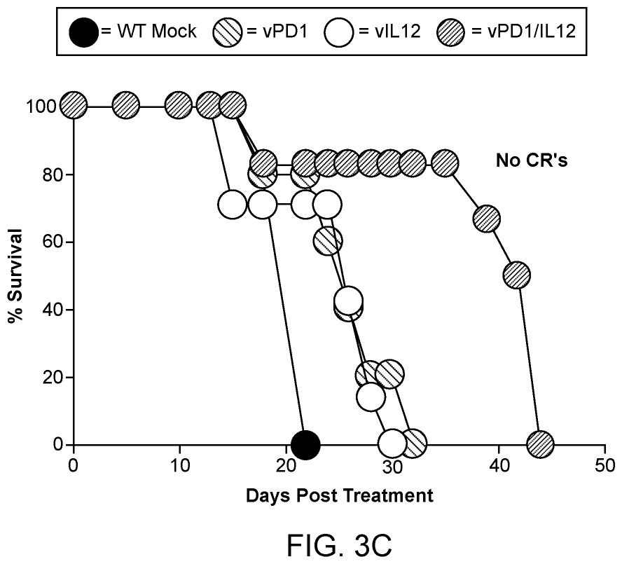

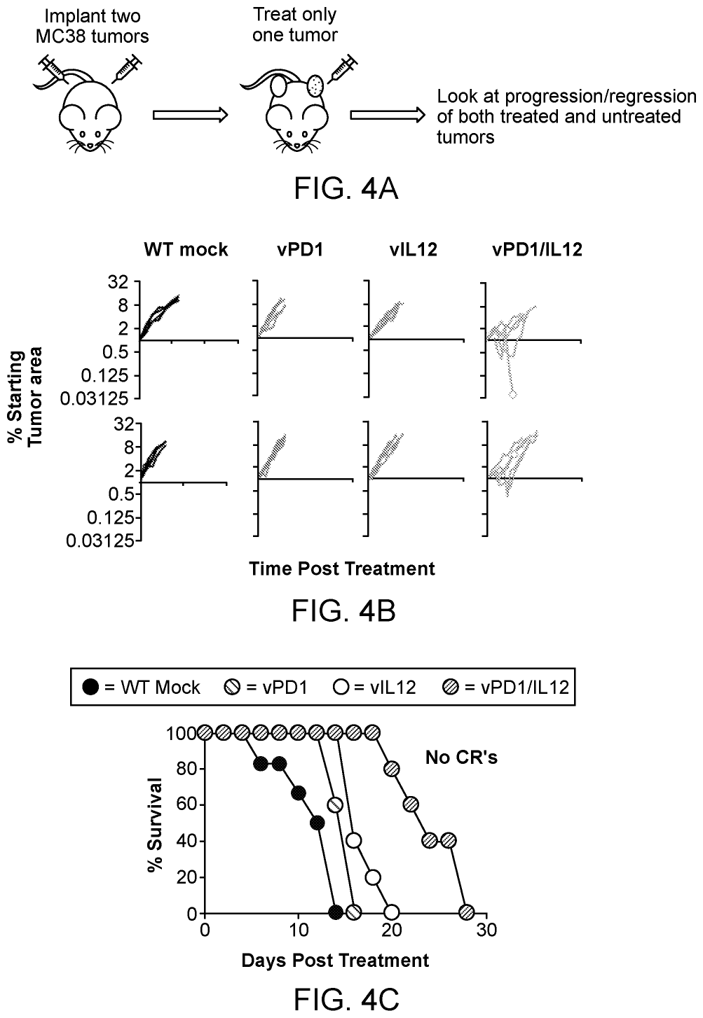

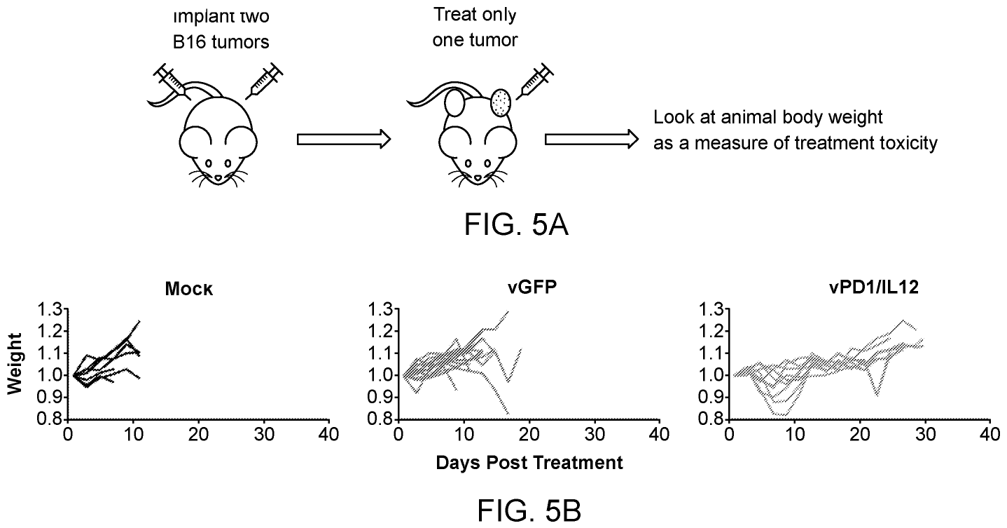

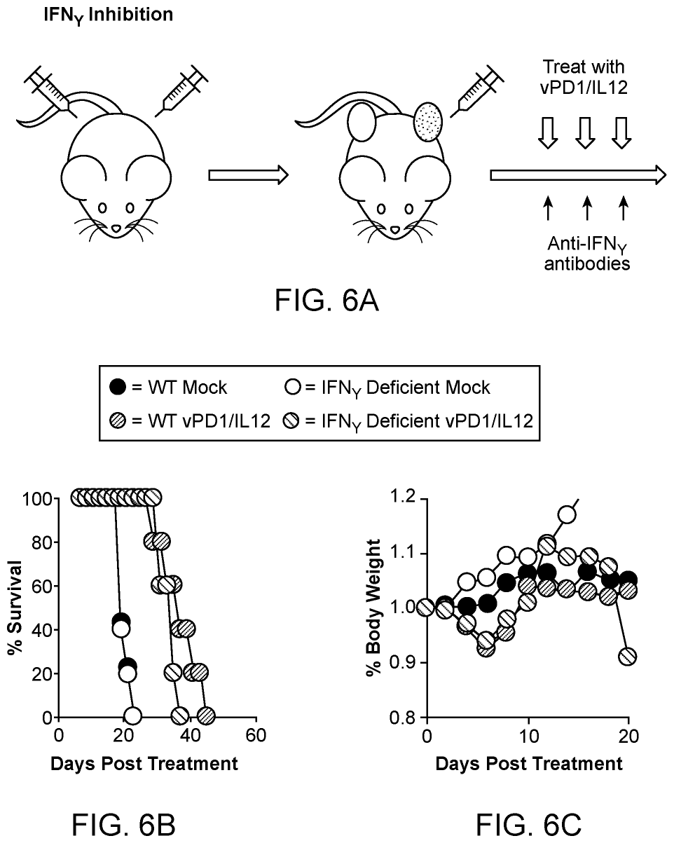

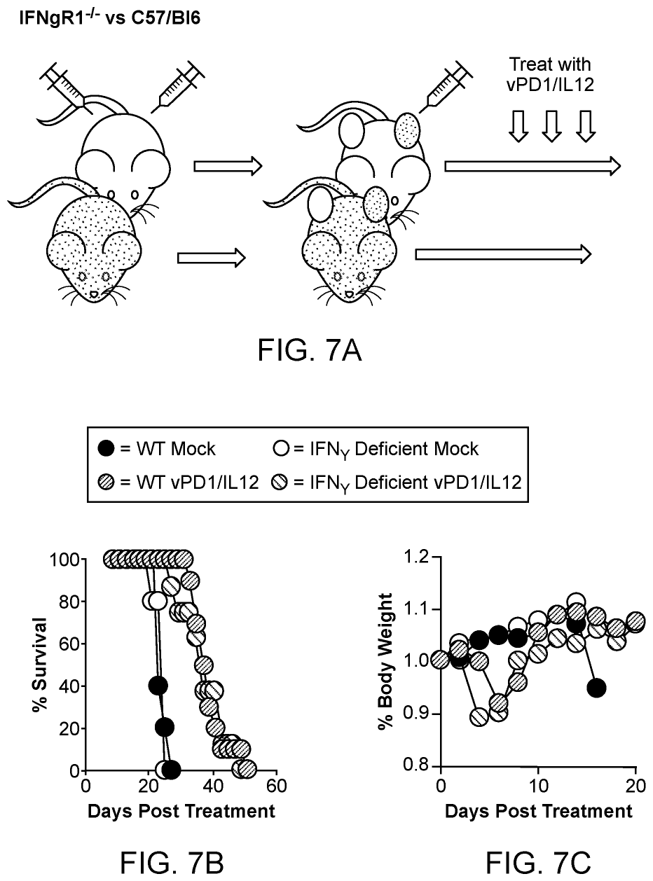

### DETAILED DESCRIPTION OF ILLUSTRATIVE EMBODIMENTS

This disclosure describes a therapeutic method for treating cancer. The method combines two different therapeutic strategies, oncolytic virotherapy (OV) and tumor necrosis factor (TNF) inhibition.

Oncolytic virotherapy works by inducing localized inflammation, leading to the development of an anti-tumor immune response. Part of this inflammatory response includes induction of tumor necrosis factor (TNF) expression. TNF is expressed by numerous types of immune cells including, but not limited to, myeloid cells and CD8⁺ T cells. TNF is generally considered to be a proinflammatory cytokine and can be directly cytolytic under certain conditions. TNF induction is therefore considered a positive event in tumor immunotherapy. For example, direct expression of TNF from an oncolytic virus can be beneficial to immunotherapy (Beug et al., 2018, Mol Ther Oncolytics 10:28-39; Havunen et al., 2018 Mol Ther Oncolytics 11:109-121; Havunen et al., 2017, Mol Ther Oncolytics 4:77-86; Cervera-Carrascon et al., 2021, Front Immunol 12:706517; Heiniö et al, 2020, Cells 9 (4): 798).

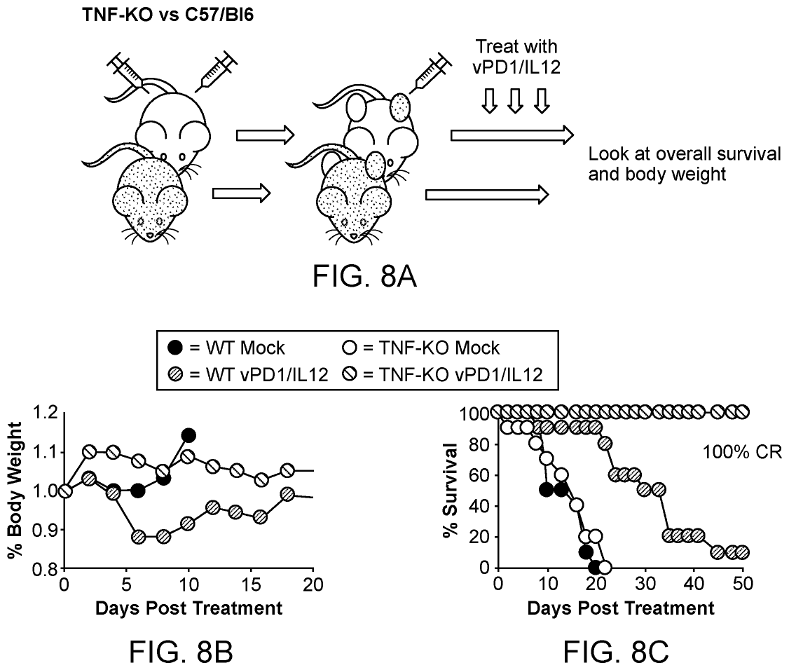

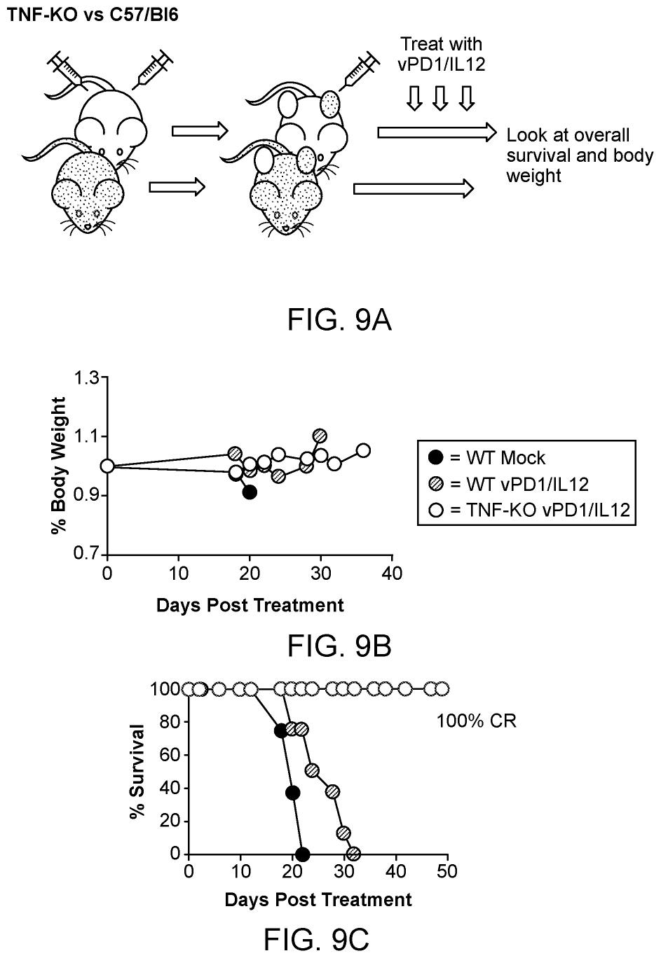

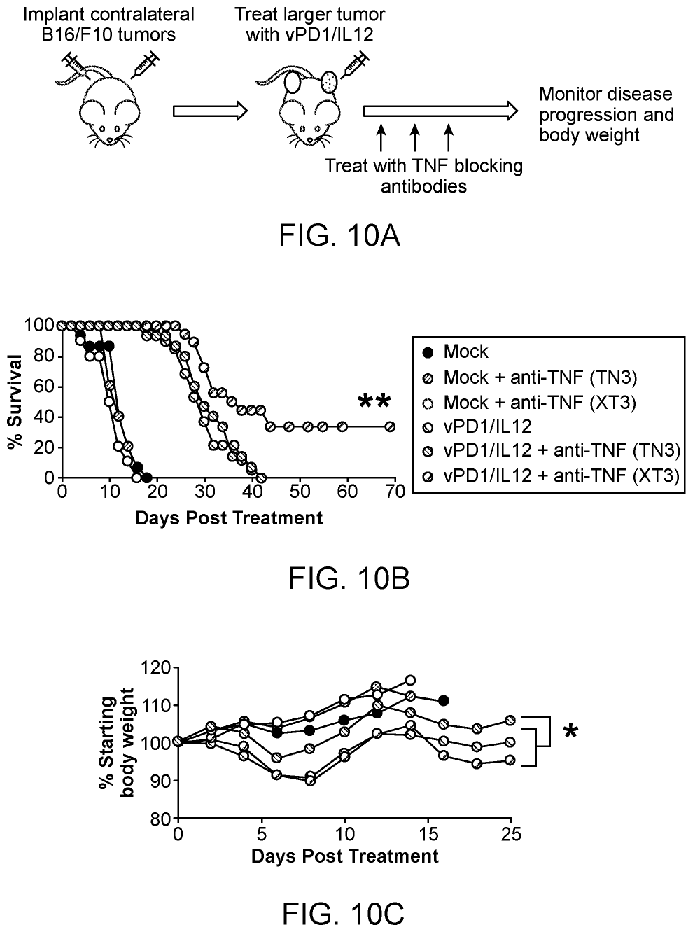

In one aspect, this disclosure describes tumor regression caused by the oncolytic myxoma virus vPD1/IL12 and inhibition of interferon-γ (IFN-γ) and/or tumor necrosis factor (TNF). IFN-γ and TNF are antiviral cytokines typically expressed during an inflammatory response. IFN-γ is a potent effector molecule expressed by Tₕ1 helper T cells, CD8⁺ T cells, and natural killer (NK) cells. IFN-γ has proinflammatory activity and is generally involved in T-cell-mediated immunotherapy. TNF is a signaling molecule produced by immune cells such as macrophages in response to an inflammatory stimulus. Induction of TNF during oncolytic virotherapy is generally viewed as beneficial, as inflammation often leads to cell death, which may further tumor reduction. Unexpectedly, as described herein, genetic loss of TNF using TNF knockout mice, caused an increase in the efficacy of viral therapy. Complete cure rate went from 0% to 100% in two different models using TNF knockout mice (FIG. 8, FIG. 9). Extrapolating this observation to clinical inhibition of TNF generated qualitatively similar results. In one exemplary animal model, oncolytic viral therapy in combination with a systemically administered TNF inhibitor (e.g., an anti-TNF antibody) improved survival from 0% to 60% (FIG. 10).

In one or more embodiments, genes of interest are incorporated into an expression cassette packaged by the oncolytic virus. The expression cassette may include one or more gene expression elements including, but not limited to, at least one promoter, at least one polyadenylation sequences, at least one regulatory element, or at least one other expression element. The expression cassette may be directly incorporated into the viral genome, either adding to the genome or replacing a viral gene. In one or more embodiments, the expression cassette may include an expression marker such as a fluorescent or bioluminescent protein.

PD-1 (programmed cell death protein 1) is a protein typically expressed on the surface of T cells and B cells. PD-1 promotes immune self-tolerance by recognizing “self” ligands and suppressing inflammation.

In one or more embodiments, the expression cassette may include a portion of the full PD-1 gene. In one or more of these embodiments, the portion of the full PD-1 gene may be the soluble ectodomain of PD-1. As used herein, “vPD1” refers to the soluble ectodomain of PD-1. As used herein, the “soluble ectodomain” refers to the extracellular portion of a mature PD-1 protein. Alternately, the expression cassette may include the full PDCD1 coding sequence. The PDCD1 gene used may be derived from any suitable source. The PD-1 gene used may be selected to match the treated subject, e.g., human PD-1 may be used to treat a human subject, and murine PD-1 may be used to treat a mouse subject. In one or more embodiments, the expression cassette may include at least one additional transgene. The additional transgene may be a checkpoint inhibitor or a proinflammatory cytokine, such as an interleukin. Suitable checkpoint inhibitors include LAG3, TIM3, VISTA, BTLA, CTLA4, or TIGIT. In one or more embodiments, the additional transgene is IL-2, IL-25, IL-28, or IL-12. The additional transgene may include the full interleukin protein, or it may include only a portion of the interleukin. In some preferred embodiments, the additional transgene includes the IL-12a (p40) and IL-12b (p35) domains of IL-12 expressed as a fusion protein with a flexible linker.

FIG. 10 shows the experimental design and results using a melanoma model in mice. B16/F10 melanoma cells were implanted into both flanks of syngeneic mice and the mice were divided into three groups: mock (untreated), mice treated with the vPD1/IL12 oncolytic virus alone, and mice treated with co-administered vPD1/IL12 and a TNF inhibitor. Once the tumors reached approximately 30 mm², the larger tumor was treated (in the treatment groups) with three intratumoral injections of vPD1/IL12. Mice in the vPD1/IL12 alone group received intraperitoneal injections of isotype antibodies every other day throughout the duration of the experiment. Mice in the vPD1/IL12+anti-TNF group received intraperitoneal injections of anti-TNF antibodies every other day throughout the duration of the experiment. Progression of both the injected and contralateral tumors was monitored through day 30. Contralateral tumors progressed in the vPD1/IL12 treatment group while contralateral tumors in vPD1/IL12+anti-TNF treatment group remain well controlled. Body weight of each animal was measured every other day as an indicator of treatment toxicity. Animals in the vPD1/IL12+anti-TNF treatment group maintained their weight, indicating that the vPD1/IL 12+anti-TNF treatment was well tolerated.

Thus, this disclosure describes methods for treating a tumor in a subject. Generally, the method includes co-administering to a subject an oncolytic virotherapeutic and a TNF inhibitor in amounts that, when co-administered, are effective to ameliorate at least one symptom or clinical sign of the tumor being treated.

The subject can be a human or a non-human animal such as, for example, a livestock animal or a companion animal.

As used herein, “co-administered” refers to two or more components of a combination administered so that the therapeutic effects of each component can work in concert with the therapeutic effects of the other component(s). Two components may be co-administered simultaneously or sequentially. Simultaneously co-administered components may be provided in one or more pharmaceutical compositions and, accordingly, may be administered at the same or different sites. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered and are able to reach the treatment site. Thus, a co-administered combination can, in certain circumstances, include components that never exist in a chemical mixture with one another.

In one or more embodiments, therefore, the oncolytic virotherapeutic and the TNF inhibitor may be separately co-administered simultaneously or sequentially. In some cases, however, the TNF inhibitor may be encoded by and expressed by the oncolytic virotherapeutic.

As used herein, “ameliorate” refers to any reduction in the extent, severity, frequency, and/or likelihood of a symptom or clinical sign characteristic of a particular condition. “Sign” or “clinical sign” refers to an objective physical finding relating to a particular condition capable of being found by one other than the patient, while “symptom” refers to any subjective evidence of disease or of a patient's condition.

Generally, oncolytic virotherapeutics not only kill tumor cells, but also cause the release of danger signals that induce an immune response against the tumor. An exemplary oncolytic virotherapeutic is the myxoma virus is described in U.S. Patent Publication No. 2021/0196771 A1. In one or more embodiments in which the oncolytic virotherapeutic is the myxoma virus, it is a Lausanne strain myxoma virus. Other exemplary oncolytic virotherapeutics include, but are not limited to, virotherapeutic agents having a herpes simplex virus (HSV) backbone, adenovirus backbone, a vaccinia backbone, a reovirus backbone, an echovirus backbone, or a poxvirus backbone. Thus, exemplary suitable oncolytic virotherapeutics include, but are not limited to, talimogene laherparepvec, RIGVIR (Rigvir Ltd., Riga, Latvia), ONCORINE (SunWay Biotech Co. Ltd., Shanghai, China), pelareorep, NTX-010 (Neotropix, Inc., Malvern, PA), Coxsackievirus A21 (Viralytics Ltd., Sydney, Australia), OncoVEXGM-CSF (BioVex Inc., Woburn, MA), G207 (MediGene AG, Planegg, Germany), NV1020 (MediGene AG, Planegg, Germany), HSV1716 (Virttu Biologics, Leeds, United Kingdom), MV-CEA (Mayo Clinic, Rochester, MN), PV701 (Wellstat Therapeutics Corp., Gaithersburg, MD), MTH-68H (Hadassah Medical Organization, Jerusalem, Israel), SVV-001 (Neotropix, Inc., Malvern, PA), and JX-594 (Jennerex Biotherapeutics, San Francisco, CA).

In one or more embodiments, the inhibitor of TNF can be any compound or composition that inhibits a cellular activity induced by TNF. As used herein “TNF” most commonly refers to TNFα, also known as “cachectin”. However, the compositions and methods described herein may be applicable to other members of the TNF superfamily. TNF inhibitors include monoclonal antibodies, TNF-binding fragments thereof, or fusion proteins (e.g., chimeric proteins) that contain a TNF-binding fragment of an anti-TNF monoclonal antibody. TNF inhibitors also include single-domain antibodies, such as those isolated from camelids. In one or more embodiments, the antibody may be human or may be humanized. The inhibitor of TNF may be a full length TNF receptor, such as CD120a or CD120b, or a fragment thereof that binds to TNF. In one or more particular embodiments, the TNF receptor may be a receptor fusion protein; the TNF receptor or a fragment of the TNF receptor may be fused to another protein to impart desired qualities, such as, but not limited to, improved bloodstream circulation, recognition by the immune system, or stability. Exemplary TNF inhibitors therefore include, but are not limited to, adalimumab (human IgG1κ monoclonal antibody), etanercept (recombinant fusion protein including human TNFR2:IgG1-Fc), infliximab (humanized IgG1κ monoclonal antibody), and golimumab (human IgG1κ monoclonal antibody), certolizumab (humanized TNFα monoclonal antibody Fab′ fragment).

Accordingly, a nucleic acid sequence that encodes a TNF inhibitor (e.g., anti-TNF antibody as defined herein below, a TNF-binding fragment of a TNF inhibitor, or a fusion polypeptide that includes any form of a TNF inhibitor) can be incorporated into the nucleic acid sequence that encodes the oncolytic virotherapeutic so that the TNF inhibitor is co-expressed with proteins of the oncolytic virotherapeutic.

As used herein, the term “antibody” refers to a molecule that contains at least one antigen binding site that immunospecifically binds to a particular antigen target of interest. The term “antibody” thus includes but is not limited to a full length antibody and/or its variants, a fragment thereof, peptibodies and variants thereof, monoclonal antibodies (including full-length monoclonal antibodies), multispecific antibodies (e.g., bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Thus, as used herein, the term “antibody” encompasses antibody fragments capable of binding to a biological molecule (e.g., TNF) or a portion thereof, including but not limited to an Fab, an Fab′, an F(ab′)₂, a pFc′, an Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

Pharmaceutical compositions, whether containing the virotherapeutic agent and/or the inhibitor of TNF, may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the virotherapeutic agent and/or the inhibitor of TNF without causing any undesirable biological effects or interacting in a deleterious manner with any of the other components of the pharmaceutical composition in which it is contained.

The virotherapeutic agent may therefore be formulated into a pharmaceutical composition. The pharmaceutical composition may be formulated in a variety of forms adapted to a preferred route of administration. Thus, a composition can be administered via known routes including, for example, oral, parenteral (e.g., intratumoral, intradermal, transcutaneous, subcutaneous, intramuscular, intravenous, intraperitoneal, hepatic perfusion, intravesical, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the virotherapeutic agent may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation containing the virotherapeutic agent may be conveniently presented in unit dosage form and may be prepared by methods well known in the art of pharmacy. Methods of preparing a composition with a pharmaceutically acceptable carrier include the step of bringing the virotherapeutic agent into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of virotherapeutic agent administered can vary depending on various factors including, but not limited to, the specific virotherapeutic agent, the weight, physical condition, and/or age of the subject, and/or the route of administration. Thus, the absolute weight of virotherapeutic agent included in a given unit dosage form can vary widely, and depends upon factors such as the species, age, weight, and physical condition of the subject, and/or the method of administration. Accordingly, it is not practical to set forth generally the amount that constitutes an amount of virotherapeutic agent effective for all possible applications. Those of ordinary skill in the art, however, can readily determine the appropriate amount with due consideration of such factors.

For example, certain virotherapeutic agent may be administered at the same dose and frequency for which the virotherapeutic agent has received regulatory approval. In other cases, certain virotherapeutic agent may be administered at the same dose and frequency at which the virotherapeutic agent is being evaluated in clinical or preclinical studies. One can alter the dosages and/or frequency as needed to achieve a desired level of oncolytic activity. Thus, one can use standard/known dosing regimens and/or customize dosing as needed.

In one or more embodiments, the method can include administering sufficient virotherapeutic agent to provide a dose of, for example, from about 1×10³ infectious units (measured in foci forming units (FFU) to about 1×10¹⁰ FFU to the subject, although in one or more embodiments the methods may be performed by administering the virotherapeutic agent in a dose outside this range. In some of these embodiments, the method includes administering sufficient the virotherapeutic agent to provide a dose of from about 1×10⁶ to 1×10⁷ FFU.

A single dose may be administered all at once, continuously for a prescribed period of time, or in multiple discrete administrations. When multiple administrations are used, the amount of each administration may be the same or different. For example, a dose of 1×10⁷ FFU per day may be administered as a single administration of 1×10⁷ FFU, continuously over 24 hours, as two or more equal administrations, or as two or more unequal administrations. When multiple administrations are used to deliver a single dose, the interval between administrations may be the same or different.

In one or more embodiments, the virotherapeutic agent may be administered, for example, from a single dose to multiple doses per week, although in one or more embodiments the method can involve a course of treatment that includes administering doses of the virotherapeutic agent at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In certain embodiments, virotherapeutic agent may be administered from a single dose to a daily dose, although the methods described herein can be practiced administering the oncolytic virotherapeutic more frequently. In one or more embodiments, a course of treatment can include from one to seven doses of the oncolytic virotherapeutic per week (typically, but not limited to, one dose per day), such as, for example, two doses per week, three doses per week, or five doses per week. As one example, a course of treatment involving three doses of the oncolytic virotherapeutic per week may involve administering a dose of the oncolytic virotherapeutic to the subject on Monday, Wednesday, and Friday.

In one or more embodiments, the methods described herein can involve a treatment regimen that includes a single course of treatment or multiple courses of treatment. When multiple courses of treatment are administered to a subject, each course of treatment can involve the same dosing and administration of the oncolytic virotherapeutic or involve different dosages and/or administrations of the oncolytic virotherapeutic than any other course of treatment. In one or more embodiments, for example, a subject may receive from two to four courses of treatment with the oncolytic virotherapeutic.

As described above, the inhibitor of TNF can be provided in the same pharmaceutical composition as the virotherapeutic agent or in a pharmaceutical composition separate from the virotherapeutic agent. Embodiments in which the TNF inhibitor and the virotherapeutic agent are provided in the same pharmaceutical composition include embodiments in which the TNF inhibitor and the virotherapeutic agent are provided as separate components of a single pharmaceutical composition. However, as described in more detail, embodiments in which the TNF inhibitor and the virotherapeutic agent are provided in the same pharmaceutical composition also include embodiments in which the virotherapeutic agent includes a nucleic acid sequence that encodes the TNF inhibitor.

When provided in a pharmaceutical composition separate from the virotherapeutic agent, the TNF inhibitor may be formulated in a variety of forms adapted to a preferred route of administration. Thus, a composition can be administered via known routes including, for example, oral, parenteral (e.g., intratumoral, intradermal, transcutaneous, subcutaneous, intramuscular, intravenous, intraperitoneal, hepatic perfusion, intravesical, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the TNF inhibitor may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation containing the TNF inhibitor may be conveniently presented in unit dosage form and may be prepared by methods well known in the art of pharmacy. Methods of preparing a composition with a pharmaceutically acceptable carrier include the step of bringing the TNF inhibitor into association with a carrier that constitutes one or more accessory ingredients. In general, a formulation may be prepared by uniformly and/or intimately bringing the TNF inhibitor into association with a liquid carrier, a finely divided solid carrier, or both, and then, if necessary, shaping the product into the desired formulations.

The amount of TNF inhibitor administered can vary depending on various factors including, but not limited to, the specific TNF inhibitor, the weight, physical condition, and/or age of the subject, and/or the route of administration. Thus, the absolute amount of TNF inhibitor included in a given unit dosage form can vary widely, and depends upon factors such as the species, age, weight, and physical condition of the subject, and/or the method of administration. Accordingly, it is not practical to set forth generally the amount that constitutes an amount of TNF inhibitor effective for all possible applications. Those of ordinary skill in the art, however, can readily determine the appropriate amount with due consideration of such factors.

In one or more embodiments, a certain TNF inhibitor may be administered at the same dose and frequency for which the TNF inhibitor has received regulatory approval. In other cases, a certain TNF inhibitor may be administered at the same dose and frequency at which the TNF inhibitor is being evaluated in clinical or preclinical studies. One can alter the dosages and/or frequency as needed to achieve a desired level of TNF inhibitor. Thus, one can use standard/known dosing regimens and/or customize dosing as needed.

In one or more embodiments, the method can include administering sufficient TNF inhibitor to provide a dose of, for example, from about 10 μg/kg to about 5 mg/kg to the subject, although in one or more embodiments the methods may be performed by administering the TNF inhibitor in a dose outside this range.

A single dose of the TNF inhibitor may be administered all at once, continuously for a prescribed period of time, or in multiple discrete administrations. When multiple administrations are used, the amount of each administration may be the same or different. Thus, a dose of TNF inhibitor per day may be administered as a single administration, continuously over 24 hours, as two or more equal administrations, or as two or more unequal administrations. When multiple administrations are used to deliver a single dose, the interval between administrations may be the same or different. The manner in which the TNF inhibitor is administered to the subject is not limited so long as the TNF inhibitor is able to reach the treatment site so as to be co-administered with the oncolytic virotherapeutic as defined herein. Thus, the TNF inhibitor need not be administered simultaneously with the oncolytic virotherapeutic.

In one or more embodiments, the TNF inhibitor may be administered, for example, from a single dose to a daily dose, although in one or more embodiments the method can involve a course of treatment that includes administering doses of the TNF inhibitor at a frequency outside this range. When a course of treatment involves administering multiple doses within a certain period, the amount of each dose may be the same or different. For example, a course of treatment can include an initial loading dose, followed by a maintenance dose that is lower than the loading dose. Also, when multiple doses are used within a certain period, the interval between doses may be the same or be different. The frequency in which the TNF inhibitor is administered to the subject is not limited, so long as the TNF inhibitor reaches the treatment site so as to be co-administered with the oncolytic virotherapeutic as defined herein. Thus, the TNF inhibitor need not be administered to the subject at the same frequency as the oncolytic virotherapeutic is administered to the subject.

In one or more embodiments, the methods described herein can involve a treatment regimen that includes a single course of treatment or multiple courses of treatment. When multiple courses of treatment are administered to a subject, each course of treatment can be the same or different than any other course of treatment. In one or more embodiments, for example, a subject may receive from two to four courses of treatment with the TNF inhibitor.

The method may be used to treat any cancer treatable using a virotherapeutic agent. Exemplary cancers treatable using the compositions and methods described herein include, but are not limited to, bladder cancer, brain cancer, breast cancer, colorectal cancer, other gastrointestinal tumors, gynecological tumors, head and neck cancer, liver cancer, lung cancer, kidney cancer, melanoma, ovarian cancer, pancreatic cancer, pediatric tumors, prostate cancer, sarcoma, squamous cell carcinoma of skin, and hematologic tumors.

The methods described herein may be practiced in combination with one or more additional anti-tumor therapies. Exemplary anti-tumor therapies include, but are not limited to, chemotherapy (e.g., doxorubicin, cisplatinum, cyclophosphamide 4, etoposide, gemcitabine, ifosfamide, 5-flurouracil, leucovorin, mitomycin-C, premetrexed, codetaxel, folfox, paclitaxel, carboplatin), radiation therapy, immunotherapy (e.g., ipilimumab, pembrolizumab), prodrugs (e.g., 5-flurouracil (5-FU), ganciclovir, valganciclovir), and targeted therapy (e.g., bevacizumab, bortexomib, erlotinib, rituximab).

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

### Mouse Models

All mice used in these studies were between six and eight weeks of age. For the B16/F10, MC38, and A9 models, 1×10⁶ cells from each cell line were injected subcutaneously into the flank(s) of syngeneic C57/B16 mice. For contralateral tumor studies, cells were injected at the same time on both the right and left flanks. Treatment was initiated when both tumors reached approximately 25 mm² in area. While some experiment-to-experiment variation was observed, this was typically around day 7-9 for B16/F10 tumors and around day 14-20 for A9 tumors.

Once tumors had reached 25 mm², mice were randomly binned into the required groups and virally treated as indicated. Viral treatment consisted of three injections (given on days 0, +2, and +4). Each injection consisted of 1×10⁷ FFU of the indicated virus in 50 μl of sterile PBS and was delivered intratumorally into the larger of the two established tumors. Tumor area was then measured either every two days (for B16/F10 and MC38 tumors) or twice weekly (for A9 tumors) using digital calipers and is presented as tumor area (mm²) determined using the formula (area=length×width). For survival studies, animals were euthanized when the total area of their tumors combined to exceed 400 mm². Toxicity was measured by measuring animal body weight every other day and normalizing to starting weight.

For the peritoneal BR5 model, 1×10⁶ BR5-luciferase cells were injected interperitoneally into syngeneic FVB mice. Treatment was initiated ten days post implantation and consisted of three IP injections of 1×10⁷ FFU of the indicated virus in 200 μl sterile PBS (given on days 10, 12, and 14). Tumor burden was monitored every seven days for a total of 23 days by assaying luciferase activity in individual mice. Animals were euthanized when they displayed build-up of ascites fluid which resulted in loss of body condition.

Knockout mice used in these studies include: IFNγR1-KO (C57BL/6N-Ifngr1^{tm1.1}Rds/J) and TNF-KO (B6; 129S-Tnf^{mlGkl}/J). Blocking antibodies used in these studies include anti-IFNγ (clone XMG1.2) and anti-TNF (clone XT3.11). All animal studies were approved by, and conducted under the supervision of, the institutional animal care and use committee at the Medical University of South Carolina.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
a virotherapeutic agent; and
an inhibitor of tumor necrosis factor (TNF).

2. The composition of claim 1, wherein the virotherapeutic agent comprises an oncolytic virus.

3. The composition of claim 2, wherein the oncolytic virus comprises a myxoma virus.

4. The composition of claim 3, wherein the myxoma virus is recombinant.

5. The composition of claim 1, wherein the virotherapeutic agent comprises an expression cassette encoding the soluble ectodomain of programmed cell death protein 1 (PD1).

6. The composition of claim 1, wherein the virotherapeutic agent comprises an expression cassette encoding interleukin 12 (IL-12).

7. The composition of claim 1, wherein the inhibitor of TNF comprises an antibody that binds to TNF.

8. The composition of claim 7, wherein the antibody is a TNF-binding antibody fragment.

9. The composition of claim 1, wherein the inhibitor of TNF comprises a multispecific compound comprising a TNF-binding antibody fragment.

10. The composition of claim 8, wherein the TNF-binding antibody fragment is a Fab, a Fab′, a F(ab′)₂, a pFc′, a Fd, a single domain antibody (sdAb), a variable fragment (Fv), a single-chain variable fragment (scFv), a disulfide-linked Fv (sdFv), a diabody, a bivalent diabody, a linear antibody, a single-chain antibody molecule, or a multispecific antibody compound.

11. The composition of claim 1, wherein the inhibitor of TNF comprises a TNF receptor or a fragment thereof that binds to TNF.

12. The composition of claim 1, wherein the virotherapeutic agent comprises a nucleic acid sequence that encodes the inhibitor of TNF.

13. A method of treating a tumor in a subject, the method comprising co-administering to the subject an oncolytic virotherapeutic agent and an inhibitor of tumor necrosis factor (TNF).

14. The method of claim 13, wherein the oncolytic virotherapeutic agent comprises a myxoma virus.

15. The method of claim 14, wherein the myxoma virus is recombinant.

16. The method of claim 13, wherein the virotherapeutic agent comprises an expression cassette encoding programmed cell death protein 1 (PD1).

17. The method of claim 13, wherein the virotherapeutic agent comprises an expression cassette encoding interleukin 12 (IL-12).

18. The method of claim 13, wherein the inhibitor of TNF comprises an antibody that binds to TNF.

19. The method of claim 18, wherein the antibody is a TNF-binding antibody fragment.

20. The method of claim 13, wherein the inhibitor of TNF comprises a multispecific compound comprising a TNF-binding antibody fragment.

21. The method of claim 19, wherein the TNF-binding antibody fragment is a Fab, a Fab′, a F(ab′)₂, a pFc′, a Fd, a single domain antibody (sdAb), a variable fragment (Fv), a single-chain variable fragment (scFv), a disulfide-linked Fv (sdFv), a diabody, a bivalent diabody, a linear antibody, a single-chain antibody molecule, or a multispecific antibody compound.

22. The method of claim 13, wherein the inhibitor of TNF comprises a TNF receptor or a fragment thereof that binds to TNF.

23. The method of claim 13, wherein the oncolytic virotherapeutic agent is administered intratumorally.

24. The method of claim 13, wherein the inhibitor of TNF is administered systemically.

25. The method of claim 13, wherein the method further comprises administering an additional tumor therapy to the subject.

26. The method of claim 25, wherein the additional tumor therapy comprises chemotherapy, radiation therapy, immunotherapy, or a prodrug, targeted therapy.
